(19) United States
(12) Patent Application Publication
(10) Pub. No.: US 2025/0022593 A1
(43) Pub. Date: Jan. 16, 2025

(54) METHOD AND SYSTEM FOR AUTOMATIC REMOTE PROGRAMMING OF INFUSION PUMPS

(71) Applicant: B. Braun Melsungen AG, Melsungen (DE)

(72) Inventor: Felix Kever, Kassel (DE)

(21) Appl. No.: 18/763,096

(22) Filed: Jul. 3, 2024

(30) Foreign Application Priority Data
Jul. 11, 2023 (EP) 23184826.8

(51) Int. Cl. G16H 40/67; G16H 20/17; G16H 40/20

(52) U.S. Cl. CPC G16H 40/67; G16H 20/17; G16H 40/20

(57) ABSTRACT

In a method and system for programming infusion pumps, infusion orders are transmitted from a data management system. A number of infusion pumps are connected to a backend of an infusion pump system. The data management system transmits infusion orders to the backend for intermediate storage via an interface. An infusion pump receives buffered infusion orders from the backend via a second interface. A first verification code is assigned to the respective infusion order as a unique identifier and included in transmissions. A container is assigned to the respective infusion order, which is equipped or connected with an information carrier containing the first verification code. The infusion pump is assigned an input for entering a second verification code. The second verification code is compared with the first verification code. The infusion order is executed or executable by the infusion pump when the first and second verification codes match.

### CROSS-REFERENCE TO RELATED APPLICATION

This application claims priority under 35 U.S.C. § 119 to European Application No. 23184826.8, filed on Jul. 11, 2023, the content of which is incorporated by reference herein in its entirety.

### FIELD

The present disclosure relates to a method and a system for automatic remote programming of infusion pumps by means of infusion orders provided in/from a patient data management system of a medical facility.

### BACKGROUND

The prior art is the AutoProgramming or BCMA workflow, which has been used in parts of the world for many years, where BCMA stands for Barcode Medication Administration.

An Electronic Medical Record (EMR) system or patient data management system is required in a medical facility such as a hospital, nursing home or similar. Based on a medication order created in the EMR system for a specific patient, with a specific medication and precise application specifications, this system first creates machine-readable (barcode) labels for the medication container and-if not already present-the patient.

At the patient's bedside, the nurse scans the barcoded patient wristband, the medication container that was barcoded during preparation and, finally, the barcoded infusion pump that is to be used with a scanner that is connected to the EMR system. There, the patient ID and medication ID scanned on site are now compared with the medication order on hand (correct patient? correct medication? etc.) and, if the check is successful, sent to the infusion pump, which was also scanned previously.

A further check is carried out on the pump by comparing the medication and the application specifications (dose or flow rate, volume to be infused, limits, etc.) with the data and specifications stored in the infusion pump's medication library.

Finally, the infusion pump shows the received data on the display for final confirmation; infusion therapy can then be started. The ongoing infusion data, including the order ID, is then sent back to the EMR system so that it can be automatically assigned to the corresponding medication order u and/or the patient's electronic patient record.

The disadvantages of this procedure can be summarized as follows:
A barcode scanner and a user interface to the EMR system must be available and ready for use at the bedside for each infusion therapy to be started or must be carried by each nurse.
Going through the necessary scanning processes at the patient's bedside and the respective confirmations in the EMR system is time-consuming in terms of the process alone.
Due to the physical distances between the user interface of the EMR system, the patient wristband, the medication container and the infusion pump, multiple spatial changes may be necessary, which complicates the workflow and causes further time expenditure.
The above-mentioned points lead to a low level of acceptance among both the workflow managers and the nurses/care staff, especially when it comes to a new introduction of the BCMA workflow.
The main part of the BCMA workflow (including the scanner-based verification of the infusion order) must be implemented on the EMR system side; the infusion pump system only receives the fully checked infusion order for display on/execution by the pump. As a result, only a few EMR systems currently support the BCMA workflow.
All this can ultimately result in the occurrence of medication and treatment errors to the detriment of patients, which could certainly have been avoided with the BCMA workflow.

### SUMMARY

The objective of the present disclosure is to provide a workflow (method) and an associated system (device) for simple, error-proof remote programming of infusion pumps using an EMR system, which at least partially avoids or mitigates the disadvantages of the prior art and, in particular, does not require time-consuming scanning processes by the nursing staff at the patient's bedside.

Accordingly, a method is provided for automatically remotely programming infusion pumps by means of infusion orders provided in/from a patient data management system of a medical facility, wherein the medical facility
has a number of beds that can each be occupied by one patient,
has a number of infusion pumps, each of which is connected to a backend of an infusion pump system,
   whereby
the patient data management system transmits infusion orders to the backend for intermediate storage (buffering) via an initial electronic interface,
the respective infusion pump receives buffered infusion orders from the backend via a second electronic interface,
   whereby furthermore
a human-readable short confirmation code is directly assigned to the respective infusion order as a unique identifier and included in transmissions,
a medication container is assigned to the respective infusion order, which is equipped with an information carrier containing the assigned short confirmation code,
the respective infusion pump has a receptacle or a connection for one of the medication containers,
the respective infusion pump is assigned an input means for entering the associated short confirmation code, which can be taken from the information carrier of the medication container,
   whereby finally
in a verification step, a short confirmation code entered at the input means is compared with the short confirmation code directly associated with the infusion order received by the infusion pump, and wherein the infusion order is executed by the infusion pump or can be executed on request only if the short confirmation codes match.

In the present application, the term “bed” or “bedside” is preferably to be understood in a broad sense and includes any infusion site where an infusion can be administered to a patient or client.

This provides a significantly simplified workflow for secure remote programming of infusion pumps using an EMR system. By eliminating up to three scanning processes and shifting all user interaction to the infusion pump, the entire process is much faster, more user-friendly and easier to implement-while retaining all the benefits of the classic AutoProgramming/BCMA workflow, in particular the exclusion of incorrect assignment of medication to the patient.

In a basic version, it is necessary that the EMR system contains a (current) assignment of beds to patients and/or that the backend contains an assignment of beds to infusion pumps. Both can be implemented in the form of assignment tables, for example.

In other words, in the basic version, an error-free assignment of patient and bed location (in the EMR system) and of bed location and pump (in the infusion pump system) that is up-to-date at the time the infusion order is transmitted is a mandatory prerequisite for the procedure described.

An alternative would be the additional entry of a second, patient-related code on the pump (i.e. a code that is preferably also greatly shortened by alphanumeric coding), which is created and managed by the EMR system and identifies the patient. This code would either be additionally printed on the patient wristband or would replace the usually quite long and purely numerical patient ID. As a consequence, this would be an additional input step for the nurse, but would eliminate the need to assign the pump to a bed location.

Ideally, the comparison of the short confirmation codes takes place decentrally, outside the EMR and outside the backend, preferably in a controller integrated into the respective infusion pump. Alternatively or additionally, the comparison could also take place in the backend/in the IOM.

Furthermore, it is preferred that the respective infusion pump queries and receives infusion orders assigned to it from the backend via pull communication via the second electronic interface, while the patient data management system preferably transmits infusion orders to the backend via push communication via the first electronic interface. In the former case, this is typically an indirect assignment, as the medication orders are preferably not explicitly assigned to a pump, but to the patient, to whom the pump is in turn assigned indirectly (i.e. via the bed).

In an advantageous variant, a validity period or a validity duration is assigned to the respective infusion order and considered in the verification step. This means that a relatively short confirmation code can be sufficient even for a comparatively large medical facility with many infusions.

If the short confirmation code is an alphanumeric code, it is particularly easy for the nursing staff to handle and enter via the input field of the infusion pump. The term “alphanumeric” should include both the possibility of a purely numeric code (in particular from the digits 0 to 9) and a purely letter-based code (preferably from the Latin capital letters A to Z, possibly without O and/or without I) as well as a mixed or combined code from the aforementioned components. In the latter case, a three-digit alphanumeric code can represent around 42,000 variants and is therefore generally sufficient-also in view of the limited period of validity.

Instead of or in addition to an input via an input field/touch display on the infusion pump, the code can be entered via an input means assigned to the infusion pump, e.g. an input field on the rack, or via a mobile device (smartphone, tablet or similar).

In a preferred simple case, the information carrier is a readable label, in particular a printed label, for example in the form of a sticker labeled with the short confirmation code, which is stuck to the medication container, or in the form of a labeled slip of paper, strip, label or other labeled medium, which is connected to the medication carrier. However, it is also conceivable that the information carrier contains the short confirmation code in another form and is, for example, read by machine/automatically and fed to the input means.

The features and advantages mentioned for the method apply analogously to the device.

The system concept/workflow for the verified administration of infusion processes or Verified Infusion Administration (VIA) described in the context of the present disclosure fulfills the same purpose as the known BCMA workflow, but is much more user-friendly and therefore easier to implement and use on a daily basis.

The main differences are the control of the entire process via the infusion pump using combined push/pull communication and the elimination of all scanning processes-on the one hand by using existing data and on the other hand by using a short, human-operated confirmation code or verification code. The VIA concept requires some innovations on the part of both the EMR system and the infusion pump system.

### VIA-Related Innovations

Instead of classic barcodes (one-dimensional) or QR codes (two-dimensional), an infusion order or infusion order-specific, alphanumeric short confirmation code or short verification code (SVC) is preferred, which can be easily read by the nurse from the label of the medication container and entered via the user interface of the pump.
With a preference for just three alphanumeric characters (Latin capital letters without “O” and digits from 0 to 9), the SVC offers around 42,000 code variants, which should be sufficient to create a unique code for each infusion order, at the latest if the validity period is limited. This means that there is no need for a scanner or other reading device (e.g. smart device) when verifying the medication container.

### VIA-related Innovations on the EMR System Side

The infusion order only contains an unspecific pump ID that activates the VIA mode, as the entire process is controlled via the pump selected by the user and the pump therefore no longer needs to be actively addressed in advance.
For the verification of the medication container, a unique, three-digit SVC is generated and added to the infusion order either in addition to or instead of the order ID provided in the BCMA process.
Optionally, the infusion order can be given an earliest start time and/or a limited validity period, which must be evaluated accordingly by the receiving infusion pump system.
A spatial or logical restriction is also conceivable, e.g. to a specific care unit or a specific user group (nurse on duty, nurses of the respective ward, etc.).
In this case, the permitted characteristics must also be included in the infusion order and an additional, nurse-specific SVC must be entered at the pump as part of the order verification.

### VIA-Related Innovations in the Infusion Pump System

If the infusion order sent by the EMR system contains a special pump ID that triggers VIA mode, it is temporarily stored in an additional system component, the Infusion Order Manager (IOM), in a type of infusion order backlog.
The user can actively trigger pull communication at each infusion pump to retrieve infusion orders for the bedside or patient.
The Infusion Order Manager recognizes when pull communication is triggered on a pump/a pump is set to receive mode for infusion orders. For a given bed location assignment of the pump, the IOM then automatically sends an existing infusion order assigned to this bed location to precisely this pump.
After receiving the infusion order, the pump prompts the user to enter the medication SVC and compares it with the SVC contained in the infusion order received from the IOM. If the check is successful, all infusion order data is shown on the pump display for final confirmation, otherwise the process is aborted with a corresponding error message.
If the Infusion Order Manager has several infusion orders for a bed location, a list selection of these orders is generated, taking into account any time restrictions (earliest start time not yet reached? Permitted time window expired?) and displayed on the pump switched to ready to receive. After selecting an infusion order via the user interface of the pump, the Infusion Order Manager sends this order to this pump. If the available infusion orders are called up on another pump assigned to the same bed location, the infusion order already sent to another pump is at best still displayed, but can no longer be selected.
The list also contains an optional delete function for infusion orders that have not yet been started (can only be executed after entering a temporarily valid delete SVC).

### Desirable Requirements

The EMR system can clearly assign the patient to a bed location at any time.
In the infusion pump system, the pumps can also be assigned to a unique bed location.
If limited to a three-digit alphanumeric code, the 42,000 or so possible values (possibly also with a limited validity period) are sufficient to reliably map the number of infusions occurring simultaneously within this validity period (depending on the number of beds, pumps and infusions per pump).

### Typical VIA Programming Sequence

1. The EMR system sends the infusion order for patient X to the infusion pump system.

2. The hospital pharmacy or the nurse prepares the appropriate medication container.

3. The hospital pharmacy or the nursing staff provide the corresponding medication container with a sticker containing the order-specific Short Verification Code.

4. The nurse places the medication container in any infusion pump assigned to patient X's current bed location and activates pull communication.

5. The infusion pump receives the infusion order and requires the Short Verification Code to be entered.

6. Alternatively: The pump receives the list of available infusion orders and shows them on the display for selection. After the appropriate selection, the pump receives the order and requests the entry of the corresponding Short Verification Code.

7. The nurse enters the code, checks the order data displayed against the display on the user interface of the EMR system (alternatively against a printout of the medication order) and starts the infusion.

### Advantages of the VIA System Concept

No scanner or smart device is required to program the pump.
Instead of performing three scans at different points and controlling them via the user interface of the EMR system, the nurse only has to enter a simple three-digit code at the pump.
The user interface of the EMR system is required at most once for the final comparison between programming and medication order.
From the perspective of both the EMR system and the hospital, implementation is much easier and faster.
If there are several infusion orders for a patient, these can be selected and executed individually via each pump that is assigned to this patient's bed location.
Infusion orders for a later time can also be sent from the EMR system to the infusion pump system and then selected and executed in the predefined time window on each pump that is assigned to the bed location of the corresponding patient.
Predefined execution sequences of infusion orders can be monitored automatically (e.g. infusion order B can only be selected once infusion order A has been started or completed).
The VIA concept continues to be based on the familiar AutoProgramming/BCMA workflow, which has been established in some parts of the world for years, with the advantage of using HL7 communication and IHE Transaction Profiles. These can be easily supplemented with the few additional details required for the VIA workflow.

### BRIEF DESCRIPTION OF THE DRAWINGS

An embodiment of the present disclosure is explained in more detail with reference to the accompanying drawings.

FIG. 1 shows a schematic overview of a medical facility in which infusions are administered to patients, with various process sequences illustrated by arrows, and

FIG. 2 shows a schematic representation of an infusion pump for use in such a device.

### DETAILED DESCRIPTION

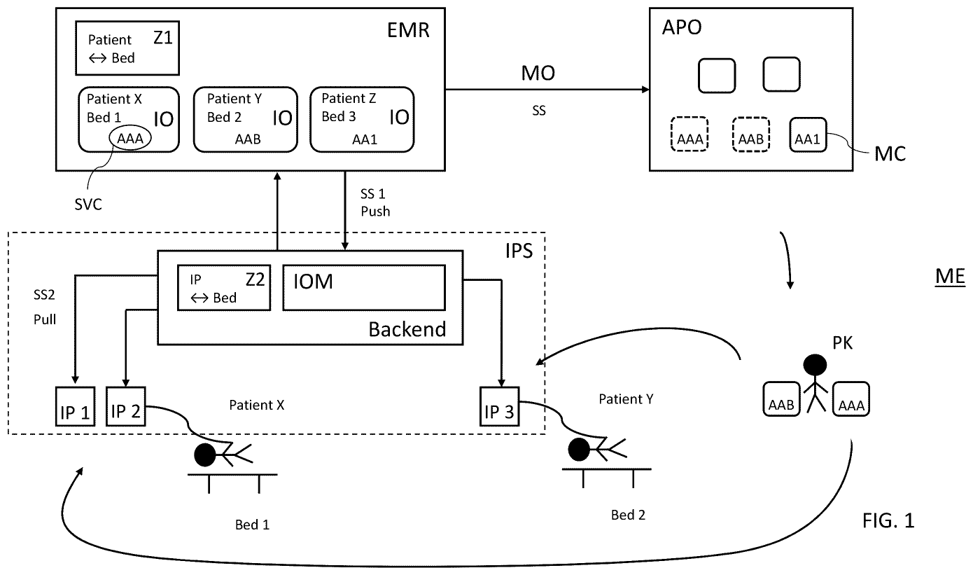

FIG. 1 provides a schematic overview of a medical facility ME in which infusions are administered to patients. This may be, for example, a hospital or a nursing home. The medical facility has a plurality of uniquely identifiable beds, whereby in this example the identification is carried out simply via a consecutive bed number (bed 1, bed 2, . . . ). In general, identification can take place via a bed location ID. Each bed can be occupied by a patient who can be identified by a unique patient ID and/or their personal data. In this example, for the sake of simplicity, these are patient X, who occupies bed 1, and patient Y, who occupies bed 2, etc. The currently valid assignment Z1 between beds and patients is stored as a data record in an electronic patient data management system (EMR—Electronic Medical Record System).

A number of infusion pumps IP is assigned to each of the beds at any given time, whereby the number can assume the values 0, 1, 2, . . . . This means that the possible cases of “no” or “several” infusion pumps IP are included, whereby each of the infusion pumps IP is assigned to exactly (and at most) one bed. The infusion pumps IP can be clearly identified by means of a pump ID, here in the example in the form of consecutive numbering (IP 1, IP 2, . . . ). In the current state shown, the infusion pumps IP 1 and IP 2 are assigned to bed 1 (and therefore to patient X), while the infusion pump IP 3 is assigned to bed 2 (and therefore to patient Y). The current assignment Z2 between beds and infusion pumps is stored as a data record in a backend of an infusion pump system IPS, which comprises the infusion pumps IP 1, IP 2, . . . .

To ensure that infusions are carried out efficiently and safely, remote programming of the assigned infusion pump IP for the specific infusion procedure in question is initiated and controlled by the EMR. For this purpose, the EMR generates a number of infusion orders (IO) according to the specifications of the attending physician. Each infusion order IO is assigned to exactly one patient in the bed occupied by that patient and contains information on the medication or infusion solution to be administered, the quantity, the flow rate, etc. Optionally, the infusion order IO can be provided with a validity date or a validity period. An administration sequence in the case of several infusions for the same patient can also optionally be integrated into the infusion order IO.

Furthermore, each infusion order IO is assigned a unique Short Verification Code (SVC), which in the example is a three-digit alphanumeric code. For example, each of the three digits can be selected from the total of the capital letters A to Z and the digits from 0 to 9, whereby the use of O (and possibly also I) is preferably omitted to avoid confusion. This results, for example, in (25+10)\hat{ }3=42,875 different labeling options, and significantly more if a four-digit code is used as an alternative and/or if lowercase letters are permitted. In this example, the infusion order IO for patient X in bed 1 is assigned the SVC “AAA”, while the infusion order IO for patient Y in bed 2 has the SVC “AAB”. The SVC can be integrated into the infusion order IO or assigned or attached as a separate element.

Medication orders MO are derived or generated from the infusion orders IO, which are transmitted via a suitable interface SS, preferably electronically, to a (hospital) pharmacy APO or to a medication supplier. In the example, the pharmacy APO prepares the required infusion solutions according to the individual specifications regarding composition, quantity, etc. and/or provides them in the form of ready-to-administer bags, syringes or the like, which are also referred to collectively as medication containers MC.

When the medication order MO is transmitted, the associated short verification code SVC is also transmitted and assigned to the respective medication container as a label, for example by attaching a correspondingly printed sticker or label. The example shows, among other things, the medication containers MC labeled with the short confirmation codes “AAA” and “AAB”, which are uniquely assigned to the infusion orders IO discussed above. In addition to the short verification code SVC, the labeling of the medication container MC can also include other information, such as that contained in the assigned infusion order IO.

The patient data management system EMR transmits the individual infusion orders IO immediately after their creation via a suitable electronic interface SS 1 using push communication to the backend of the infusion pump system IPS, where they are stored in a program module known as the infusion order manager (IOM). The transmitted infusion orders IO are available there for retrieval by the infusion pumps IP, as explained in more detail below.

For example, a nurse PK or another user takes the information from a user interface of the EMR that an infusion order IO (with the short confirmation code “AAA”) is ready for patient X in bed 1, and correspondingly for patient Y in bed 2 (with the short confirmation code “AAB”). He/she collects the medication containers MC assigned to the infusion orders IO and labeled accordingly from the pharmacy APO and brings them one after the other to the assigned beds and thus to the assigned patients. For example, the nurse knows or sees that the medication container MC with the SVC “AAA” is assigned to patient X in bed 1. The two infusion pumps IP 1 and IP 2 are available there, and if neither is occupied, the nurse usually has a free choice between the two. For example, he/she may choose the infusion pump IP 1 to carry out the infusion at bed 1. In other cases, the freedom of choice may be restricted by an existing occupancy and/or by the number of infusion pumps at the bed and/or by other criteria. For example, only the infusion pump IP 3 is available at bed 2 for carrying out the infusion order IO with the SVC “AAB”.

For example, the nurse PK inserts the medication container MC with the SVC “AAA” into the holder of the infusion pump IP 1 at bed 1 and activates pull communication by making an entry on an input field (keyboard or touch display), in which the infusion pump retrieves an infusion order IO assigned to it from the backend of the infusion pump system IPS via an interface SS 2. For example, the infusion pump IP 1 communicates its pump ID to the backend. Based on the known assignment Z2 between infusion pumps and beds, the backend recognizes that the infusion pump IP 1 belongs to bed 1 and checks the existence of infusion orders IO assigned to bed 1 in the infusion order manager IOM. If successful, the corresponding infusion order IO (here with the SVC “AAA”), or alternatively just an ID assigned to it, is transmitted from the backend to the infusion pump (here IP 1) via the electronic interface SS 2.

To verify that the correct medication container MC has been inserted into the holder of the infusion pump IP, the nurse PK is prompted via a display on the infusion pump IP to enter the SVC, which she reads from the medication container MC, via the input field. If the entry is correct (here “AAA”), the infusion pump IP is programmed according to the infusion order IO obtained or received from the backend, or a previously received infusion order IO or a completed programming is released for execution. Otherwise, an error message is displayed.

If several infusion orders are pending for bed 1 in the example mentioned, the procedure can be carried out in such a way that a selection list of the orders, possibly in combination with a sequence, is shown on the display of the infusion pump IP used, whereupon the user or nurse makes a selection and then—as described above—verifies the correct medication by entering the SVC located on the medication container and thus releases the infusion order for execution.

In a variation of the method, it can be provided that the user enters the associated SVC in the input field of the infusion pump IP directly when or after inserting the medication container MC into the holder, thereby causing the infusion pump IP to query the backend to see whether an infusion order IO assigned to this SVC is stored there. If successful, the infusion pump IP “pulls” this infusion order from the IOM in the backend. However, the selection from a list of open medication orders described above would not be possible in this case, as the selection has already been made by entering the SVC—in this respect, the list of other orders for this patient could only be displayed here for information purposes.

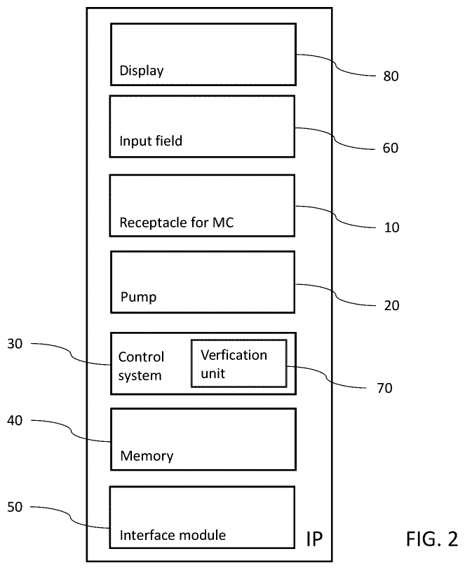

An infusion pump IP for use in the scenario described is shown schematically in FIG. 2. The infusion pump IP has a receptacle 10 for a medication container MC and a pump 20 that can be connected to the medication container MC and is controlled by an integrated control system 30. An infusion order IO individually adapted to the medication container MC and the treatment case is stored in a writable memory 40 in the manner of a control program or can be stored by up-/downloading. The infusion order IO can be received via an interface module 50 as part of the interface SS 2 via “pull” communication from a backend of an infusion pump system IPS. An associated short verification code SVC printed on the medication container MC or attached as a label is read by a nurse PK, entered via an input field 60 and then compared by a verification unit 70 of the control system 30 with the short verification code SVC directly assigned to the infusion order IO and verified if it matches. The input field 60 can be realized by a touch-sensitive keypad on a touch display, which serves to guide the user and outputs the verification result. In this case, the keypad can be specially optimized for the input of the SVC. Alternatively, a separate display 80 can be provided.

In summary, the described use and query of the short verification code SVC avoids mix-ups during transportation of the medication containers MC from the pharmacy or supplier to the assigned patient bed in a surprisingly simple way, without the need for cumbersome scanning processes or the like.

List of reference symbols

| APO | pharmacy |
|---|---|
| Backend | backend |
| Bed | patient bed |
| EMR | electronic patient data management system |
| IO | infusion order |
| IOM | infusion order manager |
| IP | infusion pump |
| IPS | infusion pump system |
| MC | medication container |
| ME | medical facility |
| MO | medication order |
| PK | nurse |
| SVC | short verification code |
| SS | interface |
| Z | assignment |
| 10 | receptacle |
| 20 | pump |
| 30 | control system |
| 40 | memory |
| 50 | interface module |
| 60 | input field |
| 70 | verification unit |
| 80 | display |

1. A method for automatic remote programming of at least one infusion pump by infusion orders provided in/from a patient data management system of a medical facility, the medical facility having a plurality of beds, and the at least one infusion pump being connected to a backend of an infusion pump system, the method comprising the steps of:
transmitting infusion orders from the patient data management system to the backend for intermediate storage via an initial electronic interface;
receiving one of the infusion orders at the at least one infusion pump from the backend via a second electronic interface;
directly assigning a first verification code that is human-readable to said one of the infusion orders as a unique identifier, the first verification code being included in transmissions;
assigning a medication container to said one of the infusion orders, the medication container being equipped or connected with an information carrier containing the first verification code, and the at least one infusion pump having a receptacle or a connection for the medication container;
entering a second verification code at an input means assigned to the at least one infusion pump, the second verification code obtainable from the information carrier; and
comparing the first verification code to the second verification code;
wherein said one of the infusion orders is executed or executable by the at least one infusion pump only when the first verification code matches the second verification code.

2. The method according to claim 1, wherein the patient data management system contains an assignment of the plurality of beds to patients.

3. The method according to claim 1, wherein the at least one infusion pump comprises a plurality of infusion pumps, and wherein the backend contains an assignment that assigns the plurality of beds to the plurality of infusion pumps.

4. The method according to claim 1, wherein the step of comparing the first verification code to the second verification code is carried out in a control system integrated in the at least one infusion pump.

5. The method according to claim 1, wherein the at least one infusion pump is configured to request infusion orders indirectly assigned to the at least one infusion pump from the backend via pull communication via the second electronic interface.

6. The method according to claim 1, wherein the patient data management system transmits infusion orders to the backend via push communication via the first electronic interface.

7. The method according to claim 1, wherein a validity period or a validity duration is assigned to said at least one of the infusion orders and is considered in the step of comparing the first verification code to the second verification code.

8. The method according to claim 1, wherein the first verification code is an alphanumeric code.

9. The method according to claim 1, wherein the medication container is labeled or marked with the second verification code.

10. A system for automatic remote programming of at least one infusion pump in a medical facility, the medical facility having a plurality of beds, each bed to be occupied by one patient, the system comprising:
a patient data management system;
an infusion pump system having a backend; and
a verification unit,
wherein:
the at least one infusion pump is connected to the backend of the infusion pump system,
the patient data management system is designed or programmed to transmit infusion orders to the backend for intermediate storage via a first electronic interface,
the at least one infusion pump is designed or programmed to receive one of the infusion orders from the backend via a second electronic interface,
a first verification code that is human-readable is directly assigned to said one of the infusion orders as a unique identifier,
a medication container is assigned to said one of the infusion orders, the medication container being equipped or connected with an information carrier containing the first verification code,
the at least one infusion pump has a receptacle or a connection for the medication container,
the at least one infusion pump is assigned an input means for entering a second verification code obtainable from the information carrier of the medication container,
the verification unit is designed or programmed to compare the first verification code to the second verification code, and
the at least one infusion pump executes, or is operable to execute upon request, said one of the infusion orders when the first verification code matches the second verification code.
